(19) United States
(12) Patent Application Publication
(10) Pub. No.: US 2021/0055254 A1
(43) Pub. Date: Feb. 25, 2021

(54) SENSOR ELEMENT AND GAS SENSOR

(71) Applicant: NGK SPARK PLUG CO., LTD., Aichi (JP)

(72) Inventor: Kazuma ITOU, Nagoya-shi, Aichi (JP)

(73) Assignee: NGK SPARK PLUG CO., LTD., Aichi (JP)

(21) Appl. No.: 16/958,297

(22) PCT Filed: Jul. 18, 2018
(86) PCT No.: PCT/JP2018/026858

§ 371 Date: Jun. 26, 2020

(30) Foreign Application Priority Data
Dec. 27, 2017 (JP) 2017-251799

(51) Int. Cl. G01N 27/407; G01N 33/00; G01N 27/409

(52) U.S. Cl. CPC G01N 27/4075; G01N 27/409; G01N 27/4077; G01N 33/0036

(57) ABSTRACT

A sensor element includes an oxygen ion conductive solid electrolyte body, a detection electrode disposed on a first surface of the solid electrolyte body for contact with a subject gas, and a reference electrode disposed on a second surface of the solid electrolyte body for contact with a reference gas. The sensor element further includes a catalyst layer which covers the detection electrode and includes a porous carrier and at least one catalyst selected from the group consisting of Ru, Rh, Pd, Ir, and Pt and supported on the carrier. The carrier includes, as a main component, aggregates of ceramic particles and Ti oxide particles different from the ceramic particles and having a smaller diameter than the ceramic particles.

### TECHNICAL FIELD

The present invention relates to a sensor element and a gas sensor for detecting the concentration of a subject gas.

### BACKGROUND ART

One known gas sensor for detecting the concentration of oxygen in exhaust gas from, for example, an automobile includes a sensor element including a detection electrode and a reference electrode which are disposed on the surface of a tubular or plate-shaped solid electrolyte. A porous electrode protection layer for protecting the detection electrode from poisoning is formed on the surface of the detection electrode.

Incidentally, in recent years, there is a need for a gas sensor which allows more accurate combustion control for an internal combustion engine, which is effective for the tightened emission regulations. This has led to demand for a gas sensor suitable for such a purpose; i.e., a gas sensor with less λ point deviation and capable of measuring the concentration of oxygen accurately. However, with conventional gas sensors, the accuracy of oxygen concentration measurement may decrease depending on, for example, the type of exhaust gas. For example, hydrogen in exhaust gas can more easily reach the detection electrode than other exhaust gas components, because hydrogen in exhaust gas is high in diffusion speed (speed at which the exhaust gas moves and reaches the detection electrode through a porous protection layer). The hydrogen reaching the detection electrode reacts with the detection electrode, causing the detection electrode to make erroneous determination. In this case, the determined λ point may deviate from the to-be-detected λ point, so that precise combustion control may be difficult to perform.

In view of the above, a technique for reducing the λ point deviation has been developed (Patent Document 1). In this technique, catalyst particles such as Pt particles are supported on the electrode protection layer to cause hydrogen in the exhaust gas to react in the porous protective layer to thereby prevent hydrogen from reaching the detection electrode.

### PRIOR ART DOCUMENT

### Patent Document

Patent Document 1: Japanese Patent Application Laid-Open (kokai) No. 2006-58282

### SUMMARY OF THE INVENTION

### Problem to be Solved by the Invention

There is a need for a gas sensor with no reduction in responsiveness. However, depending on the form of the porous layer supporting catalyst particles, gas permeability etc. may deteriorate, and this may cause a reduction in the responsiveness.

Accordingly, it is an object of the present invention to provide a sensor element which uses a catalyst to improve the accuracy of detection of a subject gas and can prevent a reduction in the responsiveness of a gas sensor and to provide the gas sensor.

### Means for Solving the Problem

In order to solve the above-described problem, a sensor element of the present invention comprises an oxygen ion conductive solid electrolyte body, a detection electrode which is disposed on a first surface of the solid electrolyte body and with which a subject gas comes into contact, and a reference electrode which is disposed on a second surface of the solid electrolyte body and with which a reference gas comes into contact. The sensor element is characterized by further comprising a catalyst layer which covers the detection electrode and includes a porous carrier and at least one catalyst selected from the group consisting of Ru, Rh, Pd, Ir, and Pt and supported on the carrier, wherein the carrier includes, as a main component, aggregates of ceramic particles and Ti oxide particles different from the ceramic particles and having a smaller diameter than the ceramic particles.

In the sensor element, the carrier has improved gas permeability, and a reduction in responsiveness of the sensor can be prevented. The reason for this is unclear. However, this may be because of the following reason. Since the plurality of Ti oxide particles roughly bonded together and forming a network structure are present in gaps between the large-diameter ceramic particles, the gaps are not clogged, and the gas permeability is not impaired.

The phrase “Ti oxide particles different from the ceramic particles” means that the ceramic particles are not Ti oxide particles. The aggregates are not formed by chemical bonding but by physical bonding (e.g., bonding by sintering).

In the sensor element of the present invention, the Ti oxide particles may include needle-shaped particles.

In this sensor element, since the plurality of Ti oxide particles are more likely to be roughly bonded in the gaps between the ceramic particles, the gas permeability is further improved.

A gas sensor of the present invention comprises a sensor element and a metallic body which holds the sensor element. The gas sensor is characterized in that the sensor element is the sensor element according to claim 1 or 2.

### Effects of the Invention

According to the present invention, it is possible to improve the subject gas detection accuracy by using a catalyst and prevent a reduction in the responsiveness of the gas sensor.

### BRIEF DESCRIPTION OF THE DRAWINGS

FIG. 1 Cross-sectional view obtained by cutting a gas sensor according to an embodiment of the present invention along a plane extending in an axial direction.

FIG. 2 Cross-sectional view showing the structures of a sensor element and a catalyst layer.

FIG. 3 Enlarged cross-sectional view showing the structure of the catalyst layer.

FIG. 4 Illustration showing a method for measuring the diameters of ceramic particles and Ti oxide particles.

FIG. 5 Graph showing a method for evaluating the responsiveness of the gas sensor.

FIG. 6 Graph showing the responsiveness of gas sensors with different carrier compositions.

FIG. 7 SEM image of the outer surface of the catalyst layer.

FIG. 8 SEM image of a cross section of the catalyst layer.

### MODES FOR CARRYING OUT THE INVENTION

An embodiment of the present invention will next be described.

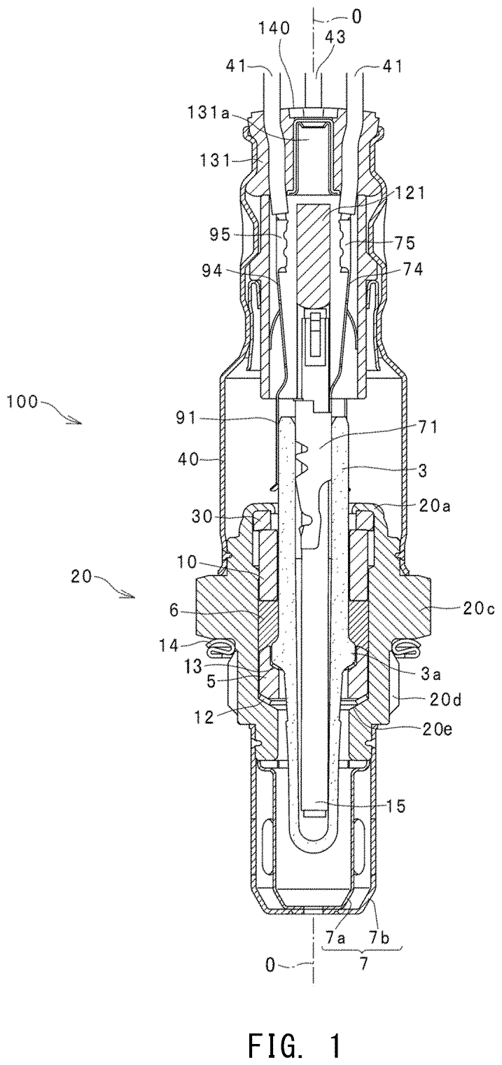

FIG. 1 shows a cross-sectional structure of a gas sensor 100 including a sensor element according to an embodiment of the present invention, the cross section being obtained by cutting the gas sensor 100 along a plane extending in the direction of an axial line O (a direction from the forward end of the gas sensor 100 to the rear end thereof). In this embodiment, the gas sensor 100 is an oxygen sensor which is to be inserted into an exhaust pipe of an automobile such that the forward end of the gas sensor 100 is exposed to exhaust gas to detect the concentration of oxygen in the exhaust gas. The sensor element 3 incorporated into the gas sensor 100 is a well-known oxygen sensor element which is an oxygen concentration cell including an oxygen ion conductive solid electrolyte body and a pair of electrodes stacked thereon and outputs a detection value corresponding to the amount of oxygen.

Notably, the lower side in FIG. 1 is referred to as the forward end side of the gas sensor 100, and the upper side in FIG. 1 is referred to as the rear end side of the gas sensor 100.

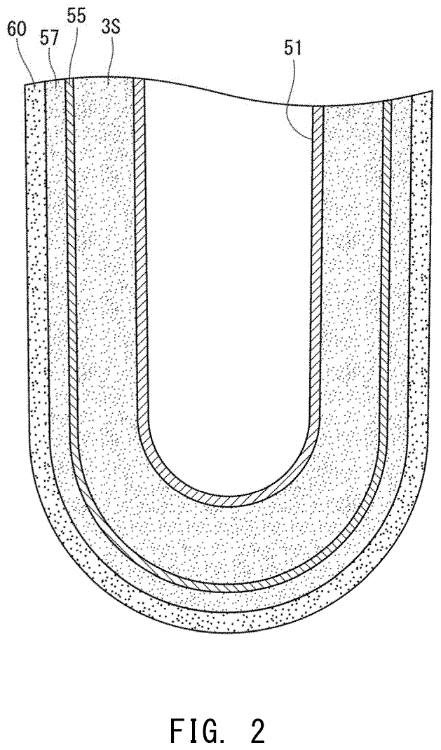

The gas sensor 100 is assembled in such a manner that an approximately cylindrical (hollow shaft-shaped) sensor element (an oxygen sensor element in this example) 3 with a closed forward end is inserted into and held by a tubular metallic body (metallic shell) 20. As shown in FIG. 2, the sensor element 3 includes: a tubular solid electrolyte body 3s tapered toward the forward end; inner and outer electrodes 51 and 55 formed on the inner and outer circumferential surfaces, respectively, of the solid electrolyte body; a catalyst layer 60 covering the outer electrode 55; etc. A round bar-shaped heater 15 is inserted into the hollow space of the sensor element 3 to heat the sensor element 3 to its activation temperature.

Notably, the outer electrode and the inner electrode correspond to the “detection electrode” and the “reference electrode,” respectively, in the claims.

A tubular outer tube 40 is joined to a rear end portion of the metallic body 20. The outer tube 40 holds lead wires 41 and terminals 74 and 94 (described later) which are disposed rearward of the sensor element 3 and covers a rear end portion of the sensor element 3. A cylindrical columnar insulating separator 121 is fixed to the inner side of a portion of the outer tube 40, the portion being located on the rear end side of the sensor element 3. A detection section at the forward end of the sensor element 3 is covered with a protector 7. A male screw portion 20d of the metallic body 20 of the thus-produced gas sensor 100 is attached to a screw hole of, for example, an exhaust pipe such that the detection section at the forward end of the sensor element 3 is exposed to the inside of the exhaust pipe, and the subject gas (exhaust gas) is thereby detected. Notably, a polygonal flange portion 20c for engagement with, for example, a hexagonal wrench is provided near the center of the metallic body 20, and a gasket 14 which prevents leakage of gas after attachment to the exhaust pipe is fitted to a step portion between the flange portion 20c and the male screw portion 20d.

A flange portion 3a is disposed near the center of the sensor element 3, and a step portion 20e whose diameter is reduced toward the inner side is provided on the inner circumferential surface of the metallic body 20 at a position close to its forward end. A tubular ceramic holder 5 is disposed on a rearward facing surface of the step portion 20e through a washer 12. The sensor element 3 is inserted into the metallic body 20 and the ceramic holder 5, and the flange portion 3a of the sensor element 3 abuts against the ceramic holder 5 from the rear end side.

Moreover, a tubular talc powder ring 6 and a tubular ceramic sleeve 10 are disposed rearward of the flange portion 3a to be located in the radial gap between the sensor element 3 and the metallic body 20. A metallic ring 30 is disposed rearward of the ceramic sleeve 10. A rear end portion of the metallic body 20 is bent inward to form a crimp portion 20a to press the ceramic sleeve 10 toward the forward end side. The talc ring 6 is thereby compressed, and the ceramic sleeve 10 and the talc powder ring 6 are fixed by means of crimping. The gap between the sensor element 3 and the metallic body 20 is thereby sealed.

Insertion holes (four insertion holes in this example) are formed in the separator 121 disposed rearward of the sensor element 3, and plate-shaped base portions 74 and 94 of inner and outer metallic terminals 71 and 91 are inserted into and fixed to two of the insertion holes. Connector portions 75 and 95 are formed at the rear ends of the plate-shaped base portions 71 and 94, and the lead wires 41, 41 are connected to the connector portions 75 and 95 by crimping the connector portions 75 and 95. Two heater lead wires 43 (only one is shown in FIG. 1) extending from the heater 15 are inserted into two unillustrated insertion holes (heater lead holes) of the separator 121.

A tubular grommet 131 is fixed, by means of crimping, to the inner side of a portion of the outer tube 40, the portion being located rearward of the separator 121, and the two lead wires 41 and the two heater lead wires 43 extend outward from four insertion holes of the grommet 131.

Notably, a through hole 131a is formed at the center of the grommet 131 and is in communication with the inner space of the sensor element 3. A water-repellent ventilation filter 140 is fitted to the through hole 131a of the grommet 131 so as to introduce a reference gas (atmospheric air) into the inner space of the sensor element 3 while preventing water from the outside from passing through the ventilation filter 140.

Notably, the tubular protector 7 is externally fitted onto a forward end portion of the metallic body 20 to cover a forward end portion of the sensor element 3 protruding from the metallic body 20. The protector 7 is configured by, for example, welding together an outer tubular protector 7b and an inner tubular protector 7a each having a plurality of holes (not shown), having a closed bottom, and made of a metal (such as stainless steel).

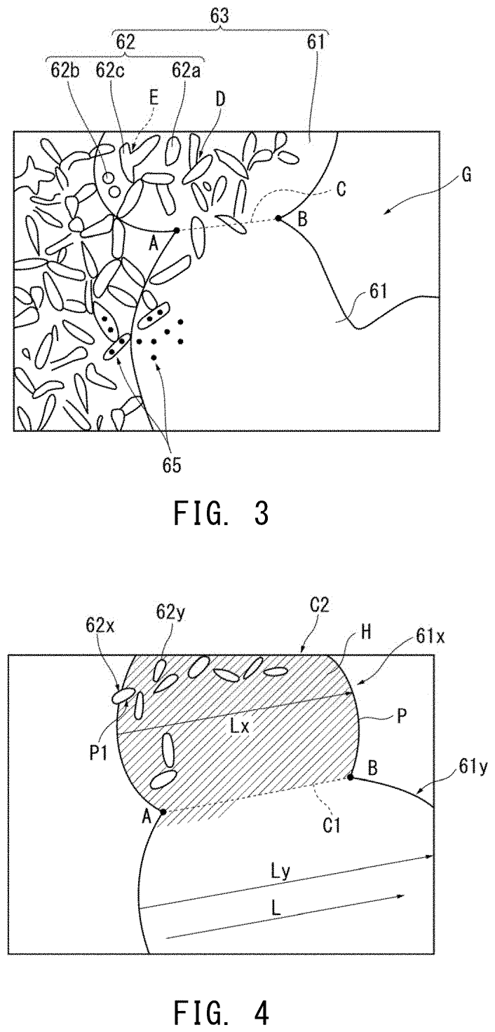

Referring next to FIGS. 2 and 3, the structures of the sensor element 3 and the catalyst layer 60 will be described. As shown in FIG. 2, the inner electrode 51 is formed on the inner circumferential surface of the solid electrolyte body 3s, and the outer electrode 55 is formed on the outer circumferential surface of the solid electrolyte body 3s. The catalyst layer 60 is formed on the surface of the outer electrode 55. A porous gas limiting layer 57 is disposed between the outer electrode 55 and the catalyst layer 60.

An additional layer (e.g., a porous protective layer) may be formed on the outer surface of the catalyst layer 60.

The gas limiting layer 57 controls the rate of gas permeation and can be formed by plasma spraying of a refractory ceramic such as alumina-magnesia spinel.

Notably, the solid electrolyte body 3s has oxygen ion conductivity and may contain, as a main component, for example, partially stabilized zirconia (YSZ) containing yttria as a stabilizer. The main component is a component contained in the solid electrolyte body 3s in an amount of more than 50% by mass.

The inner electrode 51 is exposed to a reference gas atmosphere introduced into the inner space of the sensor element 3, and the outer electrode 55 is exposed to the subject gas. Gas detection is performed between the inner electrode 51 and the outer electrode 55 through the solid electrolyte body 3s.

The inner electrode 51 and the outer electrode 55 are formed mainly of, for example, Pt. The phrase “formed mainly of Pt” means that the amount of Pt contained in an electrode is more than 50% by mass.

As shown in FIG. 3, the catalyst layer 60 includes a porous carrier 63 and at least one catalyst 65 selected from the group consisting of Ru, Rh, Pd, Ir, and Pt and supported on the carrier 63. For example, the catalyst 65 is in the form of particles, and a large number of catalyst particles are dispersed on the surface of the carrier 63.

The carrier 63 includes, as a main component, aggregates of ceramic particles 61 and Ti oxide particles 62 with a smaller diameter than the ceramic particles 61. The main component is a component contained in the catalyst layer 60 in an amount of more than 50% by mass.

Preferably, the ceramic particles 61 include at least one type of particles selected from the group consisting of, for example, alumina particles, alumina-magnesia spinel particles, and zirconia particles and are, for example, alumina-magnesia spinel particles.

The Ti oxide particles 62 are, for example, TiO₂ particles but may contain a non-stoichiometric compound containing oxygen such as TiO_{2.5}.

The thickness of the catalyst layer 60 is preferably 10 to 1,000 μm.

When the carrier 63 includes, as a main component, the aggregates (sintered aggregates) of the ceramic particles 61 and the Ti oxide particles 62 with a smaller diameter than the ceramic particles 61, the gas permeability of the carrier 63 is improved, and a reduction in the responsiveness of the sensor can be prevented.

The reason for this is unclear. However, this may be because of the following reason. Since the plurality of Ti oxide particles 62 roughly bonded together and forming a network structure are present in gaps G between the large-diameter ceramic particles 61, the gaps G are not clogged, and the gas permeability is not impaired. However, when other fine particles (such as alumina, zirconia oxide, or YSZ particles) are used, the interaction between the noble metal and the fine particles may change the catalytic ability of the noble metal, and this may affect the responsiveness, although the reason for this is unclear.

Since the carrier 63 includes the small-diameter Ti oxide particles 62, the overall surface area of the carrier 63 increases, and therefore the area of contact between the catalyst 65 formed on the carrier 63 and the subject gas increases, so that the catalytic ability may be improved. When only the Ti oxide particles 62 are used, it is difficult to form a porous carrier, so that the large-diameter ceramic particles 61 are also used.

The ceramic particles 61 and the Ti oxide particles 62 can be identified by performing elementary analysis on a cross section of a sample of the carrier 63 using an EPMA (electron probe microanalyzer) or EDS (energy dispersive X-ray spectrometer). The diameters of the ceramic particles 61 and the Ti oxide particles 62 are determined by obtaining the circle-equivalent diameters of the ceramic particles 61 and the Ti oxide particles 62 identified by the elementary analysis on the cross section of the sample of the carrier 63.

However, since a plurality of Ti oxide particles 62 are present on the surfaces of the ceramic particles 61 and in gaps G between adjacent ceramic particles 61 as shown in FIG. 3, it is difficult to determine the outlines of individual ceramic particles 61.

To reduce the influence of these Ti oxide particles 62, ten cross-sectional SEM images of different 20×20 μm viewing areas of the carrier 63 as shown in FIG. 4 are prepared.

In each of the cross-sectional SEM images, the outline P of a region H in which a ceramic particle 61x identified by the EPMA or EDS is present is extracted, and the circle-equivalent diameter of the region H is used as the diameter of the ceramic particle 61x. The average of 50 diameters arbitrarily selected from the cross-sectional SEM images is used as the diameter of the ceramic particles 61x.

Some ceramic particles 61 may be bonded and aggregated by sintering. In this case, their boundaries may be unclear.

When a ceramic particle 61x and its adjacent ceramic particle 61y are bonded by sintering as shown in FIG. 4, their boundary is determined as follows.

When the outline P of the ceramic particle 61x is narrowed in a region between points A and B to form a neck portion, a direction parallel to a straight line C1 connecting the points A and B is denoted by L. Then the longest lengths within the outlines of the mutually connected ceramic particles 61x and 61y in the direction parallel to the direction L are denoted by Lx and Ly, respectively. When the length of the line C1 between the points A and B is shorter than both Lx and Ly, the two ceramic particles 61x and 61y are regarded as being bonded as a result of sintering in the region between A and B, and the straight line C1 is defined as the boundary between the two ceramic particles 61x and 61y.

When a portion of a ceramic particle 61x is located outside an outer edge C2 of the viewing area, the outer edge C2 is regarded as a part of the outline p of the ceramic particle 61x.

When the outermost outline P of the ceramic particle 61x intersects a Ti oxide particle 62x, the outline P1 of the boundary between the Ti oxide particle 62x and the ceramic particle 61x is regarded as part of the outline P of the ceramic particle 61x. Meanwhile, Ti oxide particles 62y present inside the outline P of the ceramic particle 61x are ignored.

Therefore, the straight lines C1 and C2 are regarded as part of the outline P of the ceramic particle 61x, and the area surrounded by the entire outline P is used as the circle equivalent diameter of the ceramic particle 61x.

FIG. 3 shows regions in which Ti oxide particles 62 identified by the EPMA or EDS are present. When a clear boundary D is present between adjacent Ti oxide particles 62, the circle-equivalent diameters of the outlines separated by the boundary D are used as the diameters of the Ti oxide particles 62. When the boundary E between two Ti oxide particles 62 is unclear and these particles look as if they are bonded together, the boundary E is ignored, and the circle-equivalent diameter of the area surrounded by the entire outline is used as the diameter of the Ti oxide particles 62.

To determine the diameter of the Ti oxide particles 62, one of the cross-sectional SEM images is selected, and the diameters of all the Ti oxide particles 62 in the image are determined. The average of 50 diameters of all the diameters is used as the diameter of the Ti oxide particles 62.

When the Ti oxide particles 62 include needle-shaped particles, a plurality of Ti oxide particles 62 are more likely to be roughly bonded in gaps G between the ceramic particles 61, and this may provide higher gas permeability.

Notably, as shown in FIG. 3, the Ti oxide particles 62 may include, in addition to the needle-shaped particles 62a, spherical particles 62b and irregularly shaped particles 62c. The “needle-shaped particles” are particles with an aspect ratio of 3 or more. The aspect ratio of a Ti oxide particle 62 is the ratio of the maximum length (major axis) within the outline of the Ti oxide particle 62 to the maximum width (minor axis) in a direction orthogonal to the major axis.

The sensor element 3 of the present embodiment can be produced, for example, as follows. First, for example, yttria is added to zirconia, and the mixture is granulated, formed into a prescribed shape (see, for example, FIG. 1), and then fired at a prescribed temperature (e.g., 1,400 to 1,600° C.) to thereby produce the solid electrolyte body 3s. Then the outer electrode 55 is formed on the outer circumferential surface of the solid electrolyte body 3s using, for example, vapor deposition or chemical plating.

Notably, in this stage, the inner electrode 51 is not formed on the inner side of the solid electrolyte body 3s.

Next, a slurry prepared by mixing the ceramic particles 61, the Ti oxide particles 62, and glass powder is applied to the surface of the outer electrode 55 to form a green catalyst layer. If necessary, before or after the green catalyst layer is formed, the gas limiting layer 57 or a protective layer is formed using a routine method.

Next, the entire solid electrolyte body 3s is subjected to heat treatment in a reducing atmosphere at a prescribed temperature (e.g., 1,000 to 1,300° C.) to form the carrier 61 for the catalyst layer 60.

Next, the carrier 61 is impregnated with a solution of a noble metal used as the catalyst 65 (e.g., a noble metal complex solution) and fired to cause fine particles of the catalyst 65 to be supported on the surface of the carrier 61. Then the inner electrode 51 is formed on the inner surface of the heat-treated solid electrolyte body 3s by, for example, vapor deposition or chemical plating, and the sensor element 3 is thereby completed.

It will be appreciated that the present invention is not limited to the embodiment described above and encompasses various modifications and equivalents within the spirit and scope of the present invention.

### Examples

YSZ prepared by adding 5 mol % of yttria to zirconia was granulated and then fired to produce the solid electrolyte body 3s shown in FIG. 1. Next, the outer electrode 55 was formed on the outer circumferential surface of the solid electrolyte body 3s by electroless Pt plating.

Next, spinel was plasma-sprayed onto the surface of the outer electrode 55 to form the porous gas limiting layer 57. The inner electrode 51 was formed on the inner surface of the solid electrolyte body 3s by electroless Pt plating. A slurry for the green catalyst layer 60 was applied to the surface of the gas limiting layer 57. The slurry contained 63 wt % of spinel particles with an average diameter of 30 to 40 μm, 8 wt % of glass powder, and 29 wt % of the following fine particles with an average diameter of 0.3 μm.

The fine particles were TiO₂ particles, alumina particles, ZrO₂ particles, or YSZ particles.

Next, the entire solid electrolyte body 3s was subjected to heat treatment in a reducing atmosphere, and the carrier 63 for the catalyst layer was impregnated with a Pt solution and subjected to heat treatment. The catalyst layer 60 with fine Pt particles supported on the surface of the carrier 61 was thereby completed. A plurality of sensor elements 3 differing from one another in terms of the type of fine particles were produced. Each sensor element 3 was mounted in the manner shown in FIG. 1. Gas sensors 100 as shown in FIG. 1 were thereby obtained.

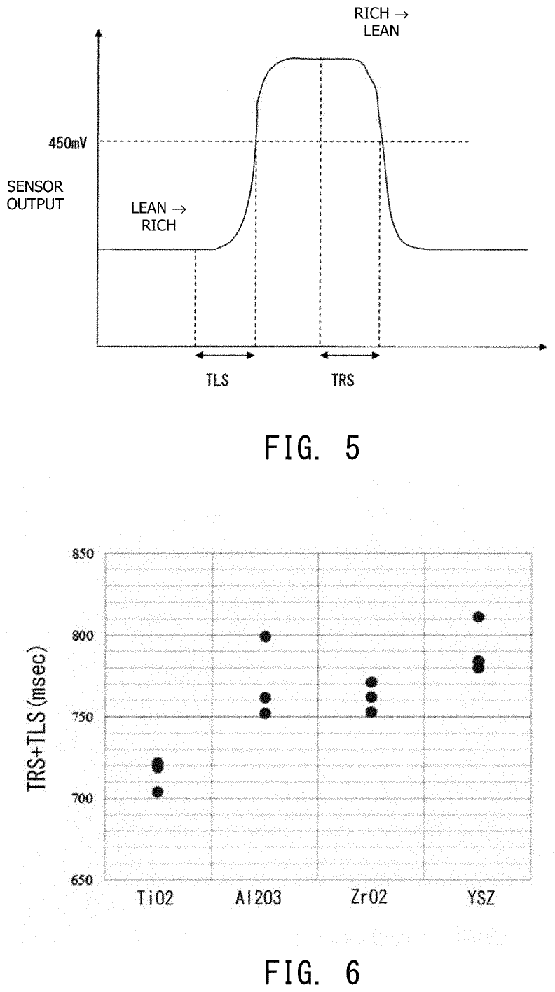

Each of the gas sensors was attached to an exhaust pipe of an engine, and the time from when engine gas (exhaust gas) was changed from lean to rich to when the sensor output became 450 mv or higher was measured as TLS, as shown in FIG. 5. Next, the time from when the engine gas was changed from rich to lean to when the sensor output became 450 mv or lower was measured as TRS.

The results obtained are shown in FIG. 6. As can be seen from FIG. 6, when the carrier contains the TiO₂ particles as well as the ceramic particles, the response time represented by (TRS+TLS) is shortest, and the responsiveness of the gas sensor is highest.

When the carrier contains alumina, ZrO₂, or YSZ particles as well as ceramic particles, the response time represented by (TRS+TLS) is longer than that when the TiO₂ particles are contained.

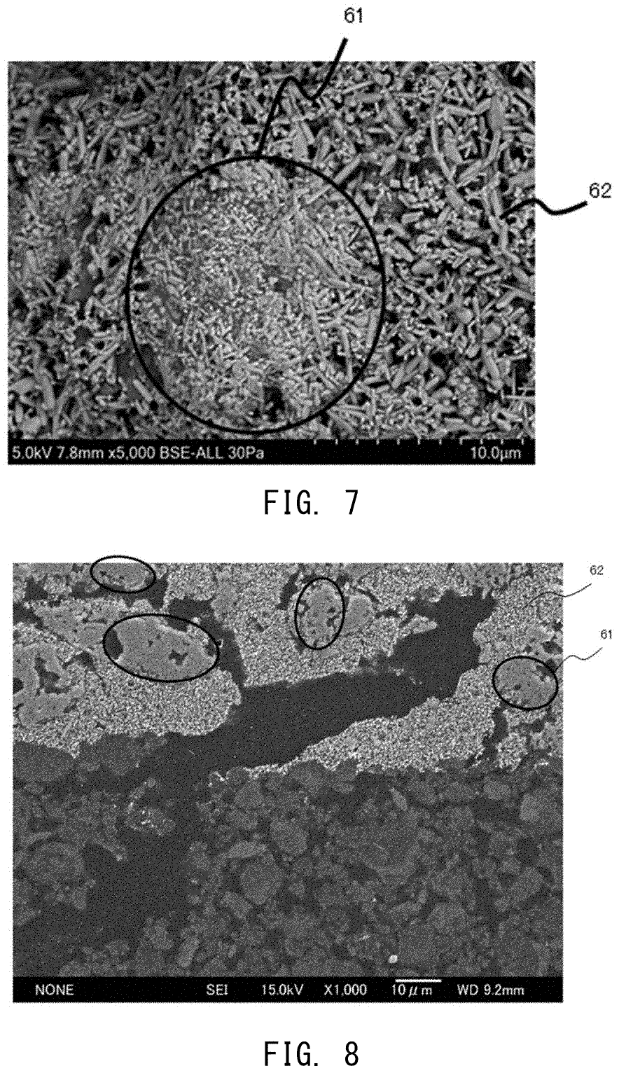

FIG. 7 shows an SEM image of the outer surface of the catalyst layer 60, and FIG. 8 shows an SEM image of a cross section of the catalyst layer 60. FIGS. 7 and 8 are secondary electron images, and circled regions in FIGS. 7 and 8 correspond to ceramic particles 61. To allow the Ti oxide particles 62 to be easily seen, each of FIGS. 7 and 8 shows the catalyst layer 60 fired with no catalyst (Pt) supported thereon.

In FIG. 8, dark portions are ceramic particles 61, and bright particulate portions (needle-shaped portions) are Ti oxide particles 62.

### DESCRIPTION OF REFERENCE NUMERALS

3 sensor element
3s solid electrolyte body
20 metallic body
51 reference electrode
55 detection electrode (outer electrode)
60 catalyst layer
61 ceramic particles
62 Ti oxide particles
63 carrier
65 catalyst
100 gas sensor

1. A sensor element comprising:
an oxygen ion conductive solid electrolyte body;
a detection electrode which is disposed on a first surface of the solid electrolyte body and with which a subject gas comes into contact; and
a reference electrode which is disposed on a second surface of the solid electrolyte body and with which a reference gas comes into contact,
the sensor element being characterized by further comprising a catalyst layer which covers the detection electrode and includes a porous carrier and at least one catalyst selected from the group consisting of Ru, Rh, Pd, Ir, and Pt and supported on the carrier,
wherein the carrier includes, as a main component, aggregates of ceramic particles and Ti oxide particles different from the ceramic particles and having a smaller diameter than the ceramic particles.

2. A sensor element according to claim 1, wherein the Ti oxide particles include needle-shaped particles.

3. A gas sensor comprising a sensor element and a metallic body which holds the sensor element, the gas sensor being characterized in that the sensor element is the sensor element according to claim 2.

4. A gas sensor comprising a sensor element and a metallic body which holds the sensor element, the gas sensor being characterized in that the sensor element is the sensor element according to claim 1.
